(19) United States
(12) Patent Application Publication
(10) Pub. No.: US 2018/0228997 A1
(43) Pub. Date: Aug. 16, 2018

(54) Method and system for gas delivery including gas conserver

(71) Applicant: L'Air Liquide, Societe Anonyme pour l'Etude et l'Exploitation des Procedes Georges Claude, Paris (FR)

(72) Inventors: Stephen Bruce Krentler, Nazareth, PA (US); Hans Irr, Nazareth, PA (US); Paul Norman Trevena, Maidenhead (GB)

(73) Assignee: L'Air Liquide, Societe Anonyme pour l'Etude et l'Exploitation des Procedes Georges Claude, Paris (FR)

(21) Appl. No.: 15/932,221

(22) Filed: Feb. 16, 2018

Related U.S. Application Data
(60) Provisional application No. 62/459,940, filed on Feb. 16, 2017.
(60) Provisional application No. 62/463,374, filed on Feb. 24, 2017.
(60) Provisional application No. 62/464,481, filed on Feb. 28, 2017.

(51) Int. Cl. A61M 16/20; A61M 16/06; A61M 16/08; B64D 11/00

(52) U.S. Cl. CPC A61M 16/207; A61M 16/0677; A61M 16/0875; B64D 2231/025; A61M 2202/0208; A61M 2205/3355; B64D 11/00

(57) ABSTRACT

A pneumatic oxygen conserver delivering a constant minute flow volume rate to a nasal mask, face mask, or nasal cannula is rendered lightweight by using tubing connected between the conserver and an oxygen source as a reservoir of oxygen for delivery to the nasal mask, face mask, or nasal cannula.

### CROSS-REFERENCE TO RELATED APPLICATIONS

This application claims priority to U.S. Provisional Application Ser. No. 62/459,940, filed on Feb. 16, 2017, U.S. Provisional Application Ser. No. 62/463,374, filed on Feb. 24, 2017, and U.S. Provisional Application Ser. No. 62/464,481, filed on Feb. 28, 2017, all of which are hereby incorporated by reference in its entirety.

### BACKGROUND

### Field of the Invention

The present invention relates to the use of gas conservers in gas delivery systems, more particularly, in gas delivery systems for inhalation of medical oxygen or emergency oxygen.

### Related Art

Gas conservers are used to provide controlled amounts of gas for inhalation by persons. Two examples of conservers include those for delivering oxygen for patient oxygen therapy and oxygen for airplane crew and/or passengers in low oxygen or low pressure environments aboard aircraft.

Oxygen conservers in the home healthcare market have for many years improved the usable duration of supply vessels (cylinders, tanks, containers, etc.) over continuous flow devices (regulators, flowmeters, liquid oxygen dewars, etc.) while still adequately satisfying the clinical needs of the oxygen patient. These systems typically only provide oxygen to the user during the inhalation portion of a breath, optimally in the first half of the user's inhalation.

Initially most devices were electronic using batteries as a power source. Later, pneumatic device using the pressure in the supply vessel as a power source became the preferred systems due to no need for batteries, smaller size and ease of use. Both types afforded the oxygen patient to ambulate for longer periods of time and/or require less frequent oxygen supply refills.

Weight reduction in aerospace is a constant goal to reduce fuel consumption, increase range and improve safety. Current systems for on-board oxygen provide continuous flow oxygen which limits supply duration, requires the maximum size supply vessel storage space will allow regardless if used which in most flights they are not and can limit aircraft range.

Some home healthcare demand systems have been tried in commercial aviation market with limited acceptance and success. Most were not durable enough for the rigors of the commercial aviation market or provided inadequate interface with the aircraft storage system.

Therefore it is an object of the invention to provide a method and apparatus for regulating gas flow with a conserver that does not experience the problems exhibited by conventional gas flow regulation methods and apparatuses.

### SUMMARY

There is disclosed a pneumatic oxygen conserving system for delivering oxygen for inhalation, comprising tubing that is adapted and configured to receive gas from a source of gas, a conserver in downstream flow communication with said tubing, and a wearable gas distribution device comprising a face mask, a nasal mask, or nasal cannula in downstream flow communication with said conserver that is adapted and configured to deliver oxygen to a person for inhalation thereof, the conserver comprising a main body into which is formed: a vent orifice that opens out of the main body; a slave chamber divided into upper and lower regions by a slave diaphragm; a sensing chamber divided into upper and lower regions by a sensing diaphragm; an inhalation sensing passageway that opens into the main body in upstream flow communication with the sensing chamber lower region; slave chamber inlet passage fluidly communicating between the main body inlet and the slave chamber upper region; a slave chamber outlet passage in flow communication between the slave chamber upper region and the mask or nasal cannula; a timing gas inlet passage in upstream fluid communication with the slave chamber lower region; and a timing gas outlet passage fluidly communicating between the slave chamber lower region, the sensing chamber upper region, and the vent orifice, wherein:

an inhalation gas flow path is comprised of said slave chamber inlet passage, said slave chamber upper region, and said slave chamber outlet passage;

a timing gas flow path is comprised of said timing gas inlet passage, said slave chamber lower region, said timing gas outlet passage, and said vent orifice;

the slave diaphragm is biased to a closed position in which it occludes flow communication between the slave chamber inlet and outlet passages and a flow of oxygen through the inhalation gas flow path is prevented;

the slave diaphragm is moved from its closed position to its open position when a difference in pressure, P_{slave upper}−P_{slave lower}, between the slave chamber upper and lower regions, respectively, exceeds a predetermined differential pressure P_{slave break};

the sensing diaphragm is biased in a closed position in which it occludes flow communication between timing gas inlet and outlet passages and prevents a flow of oxygen through the timing gas flow path;

the sensing diaphragm is moved from its closed position to its open position when a difference in pressure, P_{sensing upper}−P_{sensing lower}, between the secondary slave chamber upper and lower regions, respectively, exceeds a predetermined differential pressure P_{sensing break}; and

the conserver is adapted and configured such that:
when the sensing and slave diaphragms are in their closed positions, inhalation of a person wearing the mask or nasal cannula will cause P_{sensing upper}−P_{sensing lower} to exceed P_{sensing break} and move the sensing diaphragm to its open position, oxygen flows out of the vent orifice via the main body inlet and the timing gas inlet and outlet passages, and an oxygen flow out of the inhalation gas outlet passage is delayed by a time period ΔT₁;
   at the expiration of ΔT₁, a decrease in pressure in the timing gas flow path causes P_{slave upper}−P_{slave lower} to exceed P_{slave break} and the slave diaphragm is moved to its open position and oxygen flows through the inhalation gas flow path while the sensing diaphragms remains in its open position for a time period ΔT₂;
at the expiration of ΔT₂, P_{sensing upper}−P_{sensing lower} drops below P_{sensing break} and the sensing diaphragm is moved to its closed position and oxygen is prevented from flowing through the timing gas flow path while oxygen continues to flow through the inhalation gas flow path for a time period ΔT₃;
at the expiration of ΔT₃, an increase in pressure in the timing gas flow path causes P_{slave upper}−P_{slave lower} to drop below P_{slave break} and the slave diaphragm is moved to its closed position and flows of oxygen are prevented through the timing and inhalation gas flow pathways; and
during ΔT₂ and ΔT₃, a bolus of oxygen delivered by the conserver to the mask or nasal cannula is drawn from a reservoir in the tubing.

There is also disclosed an oxygen delivery system comprising an oxygen source, a pressure regulator, and aforementioned pneumatic oxygen conserving system. The pressure regulator is adapted and configured to regulate a pressure of the oxygen source to a lower pressure for delivery into the tubing. The tubing includes an orifice choking a flow of oxygen therethrough that separates the tubing into upstream and downstream portions. The one or both of the inhalation gas inlet and outlet passages includes an orifice choking a flow of oxygen therethrough. The downstream portion of tubing is sized and the orifice(s) of the inhalation gas inlet and outlet passages are dimensioned so as to achieve a predetermined minute volume flow rate of oxygen to the wearable gas distribution device.

There is also disclosed a method of using the aforementioned oxygen delivery system comprising the following steps. The aforementioned oxygen delivery system is provided. The oxygen distribution device is caused to be worn by a person

The pneumatic oxygen conserving system, the oxygen delivery system, and/or the method of using the oxygen delivery system may include one or more of the following aspects:
the conserver further comprises a main body inlet in fluid communication between the tubing and the inhalation and timing gas flow paths so as to receive a flow of oxygen via the tubing and the main body inlet.
the conserver further comprises a main body inlet in fluid communication between the tubing and the inhalation gas flow path and the timing gas inlet passage is in downstream flow communication with the tubing so as to receive a flow of oxygen via the tubing but not from the main body inlet.
the wearable gas distribution device is a face mask or nasal mask comprising an inspiratory valve and an expiratory valve, the conserver is integrated into the face mask or nasal mask, the inhalation gas inlet passage is in fluid communication with a downstream end of the tubing, and the inhalation gas outlet passage opens out into an interior of the face mask or nasal mask.
the wearable gas distribution device is a nasal cannula and the conserver is disposed in-line with the tubing.
the conserver is made of a plastic material.
an altitude adjustment device is disposed downstream of the tubing orifice in-line with the tubing that is adapted and configured to increase or decrease a minute volume flow rate delivered by the conserver when a decrease or increase in ambient pressure caused by an increase or decrease in altitude, respectively.
the altitude adjustment device comprises a housing having an inlet, an outlet, at least one vent holes on an end thereof that opens out to an exterior of the housing and the ambient atmosphere, and an altitude adjustment diaphragm dividing an interior of the housing into first and second regions, the first region being in flow communication with the housing inlet and outlet, the second region being in flow communication with each of the one or more vent holes, the altitude adjustment diaphragm being biased with a spring into a rest position, a decrease in ambient pressure caused by an increase in altitude causing the diaphragm to move against the biasing of the spring, decrease the volume of the second region, and increase the volume of the first region and consequently increase the volume of the tubing downstream portion.
the altitude adjustment device comprises a section of the tubing having a flexibility that is greater than remaining sections of the tubing so that a decrease in ambient pressure caused by an increase in altitude causing the section of the tubing comprising the altitude adjustment device to expand outward and increase the volume of the tubing downstream portion
the oxygen distribution device is a mask and the person is a passenger or crew member of an aircraft.
the person is a patient receiving oxygen therapy.
no other chamber is formed in the main body that is in fluid communication with the inhalation gas flow path.
no electricity is used to initiate a flow of timing gas through the timing gas flow path, initiate a flow of gas through the inhalation gas flow path, move the slave diaphragm to its open closed position, and/or move the sensing diaphragm to its open or closed position.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a pneumatic schematic visualization of the inventive conserver.

FIG. 2 is a perspective view of an embodiment of the inventive oxygen conserving system of the invention including a mask.

FIG. 3 is a perspective view of an embodiment of the inventive oxygen conserving system of the invention where the conserver is integrated into a mask.

FIG. 4 is a perspective view of an embodiment of the inventive oxygen conserving system of the invention including nasal cannula.

FIG. 5 is a perspective view of a mask into which the inventive conserver is integrated.

FIG. 6 is a perspective view of an embodiment of the inventive conserver.

FIG. 7 is a top plan view of the conserver of FIG. 6.

FIG. 8 is a top-down, perspective, exploded view of the conserver of FIG. 6.

FIG. 9 is a bottom-up, perspective, exploded view of the conserver of FIG. 6.

FIG. 10 is a cross-sectional view of the conserver of FIG. 6 taken along line I-I.

FIG. 11 is a cross-sectional view of the conserver of FIG. 6 taken along line II-II.

FIG. 12 is a perspective view of another embodiment of the inventive conserver.

FIG. 13 a top plan view of the conserver of FIG. 12.

FIG. 14 is a top-down, perspective, exploded view of the conserver of FIG. 12.

FIG. 15 is a bottom-up, perspective, exploded view of the conserver of FIG. 12.

FIG. 16 is a cross-sectional view of the conserver of FIG. 12 taken along line III-III.

FIG. 17 is a cross-sectional view of the conserver of FIG. 12 taken along line IV-IV.

FIG. 18 is a perspective view of an embodiment of the inventive oxygen conserving system of the invention including a mask and an altitude adjustment device.

FIG. 19 is a perspective view of an embodiment of the inventive oxygen conserving system of the invention including a mask and an altitude adjustment device where the conserver is integrated into the mask.

FIG. 20 is a perspective view of a first inventive altitude adjustment device.

FIG. 21 is a top plan view of the first altitude adjustment device of FIG. 21.

FIG. 22 is a cross-sectional view of the first altitude adjustment device of FIG. 21 taken along line V-V.

FIG. 23 is a cross-sectional view of a second altitude adjustment device.

### DESCRIPTION OF PREFERRED EMBODIMENTS

Together with an oxygen source, tubing, and a wearable gas distribution device (a face mask, nasal mask, or nasal cannula), the inventive gas conserver is used to supply a gas to a person only upon their inhalation. The gas is delivered in a bolus very quickly upon detection of the start of inhalation. For use in oxygen therapy, the conserver is adapted and configured for use with cryogenic oxygen systems, oxygen generators, institution wall oxygen gas outlets, portable oxygen systems, and remotely piped oxygen systems. For use aboard aircraft, the conserver is adapted and configured for use with oxygen generators (such as chemical or ceramic oxygen generators or pressure swing adsorption systems) or compressed gas cylinders containing oxygen.

A source of oxygen feeds oxygen at a regulated pressure to the tubing. The term “oxygen” should be construed to include both industrially pure oxygen and also oxygen-rich air.

The tubing includes an orifice in an upstream portion thereof. The portion of the tubing downstream of the orifice and upstream of the conservoir serves as a reservoir for the bolus of oxygen metered out by the conserver. Because this reservoir (i.e., the tubing) is not integrated into the conserver, the conserver may be made with a lower weight and smaller size. In one aspect of the invention, the minute volume flow rate delivered by the conserver may be easily adjusted by adjusting the tubing reservoir volume. Indeed, the inventive device uses the tubing downstream of the orifice as a primary reservoir chamber to accumulate and hold the bolus of oxygen for delivery to the wearer of the mask or nasal cannula until the conserver is triggered by the wearer's inspiratory breath. Along with the conserver's pneumatic timing mechanism, the inside diameter and length of the tube reservoir and the regulated pressure of oxygen supplied by the source determines the maximum volume of the bolus deliverable by the conserver. The pneumatic timing is designed to allow the maximum bolus volume to exit the tube (primary reservoir chamber).

The inventive gas conserver comprises a main body that includes a timing gas flow path and an inhalation gas (oxygen) flow path formed therein. The purpose of the timing gas flow path is to pneumatically open and close the inhalation gas flow path. More particularly, the conserver is adapted and configured such that, after an amount of time ΔT₁ after the flow of oxygen is triggered through the timing gas flow path, a flow of oxygen through an inhalation gas flow path to the mask or nasal cannula is initiated. After passage of an amount of time ΔT₂ after the flow of oxygen through the inhalation gas flow path is triggered, the flow of oxygen through the timing gas flow path is prevented, but the flow through the inhalation gas flow path continues. After passage of an amount of time ΔT₃ after the flow of oxygen through the timing gas flow path is triggered, the flow of oxygen along the inhalation gas flow path is prevented. After passage of an amount of time ΔT₄ after the flow of oxygen through the inhalation gas flow path is prevented, the pressure in the tubing reservoir reaches the regulated pressure provided by the oxygen source. After passage of ΔT₃, the cycle of oxygen delivery may be repeated when the person wearing the mask or nasal cannula draws another breath but, if the breath is drawn before expiration of ΔT₄, the size of the bolus of oxygen delivered will be prorated according to the partial portion of ΔT₄ that has passed.

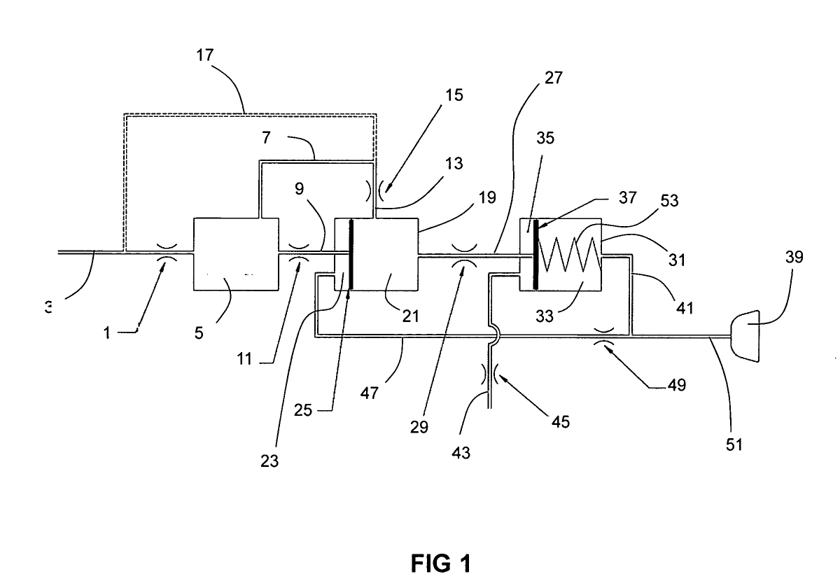

As best illustrated in FIG. 1, oxygen from a source of oxygen is received into tubing 5 via tubing inlet 3 and tubing orifice 1. The volume enclosed by the tubing 5 downstream of tubing orifice 1 and upstream of the conserver defines a reservoir of oxygen. Formed within the conserver are a slave chamber 19 and a sensing chamber 31. A slave diaphragm 25 divides the slave chamber 19 into an upper region 23 and a lower region 21. Similarly, a sensing diaphragm 37 divides the sensing chamber 31 into an upper region 35 and a lower region 33.

The tubing 5 supplies oxygen to the lower region 21 of the slave chamber 19 via a slave chamber inlet passage 7 in which is formed a slave chamber inlet orifice 15. Alternatively, the tubing 5 supplies oxygen to the lower region 21 of the slave chamber 19 via an optional slave chamber inlet passage 17. In this alternative case, the tubing 5 is provided with two lumens, one of which supplies oxygen to the lower region 21 of the slave chamber 19 and the other which supplies oxygen to the upper region 23 of the slave chamber 19 via the inhalation gas flow path inlet 9. Otherwise, the tubing 5 has just one lumen supplying oxygen to both the slave chamber inlet passage 7 (including a slave chamber inlet orifice 15) and inhalation gas flow path inlet passage 9 (including an inhalation gas flow path inlet orifice 11).

A flow of oxygen, as the timing gas, exits the lower region 21 of the slave chamber 19 via a slave chamber outlet orifice 29 disposed in a slave chamber outlet passage 27. When in its closed position, the sensing diaphragm 37 (in its closed position) occludes the slave outlet orifice 29 and prevents the timing gas from flowing out of orifice 29. When a person wearing a mask 39 inhales, the vacuum induced in passages 41, 51 and the lower region 33 of the sensing chamber 31 is sufficient to overcome the biasing of the sensing diaphragm 37 to its closed position by spring 53 and the sensing diaphragm 37 is moved to its open position and no longer occludes orifice 29. As such, the timing gas flows out of the upper region 35 of the sensing chamber and is vented out of the conserver via a sensing chamber outlet passage 43 having a sensing chamber outlet orifice 45 disposed therein. Consistent with the foregoing, one of ordinary skill in the art will recognize that the timing gas flow path includes slave chamber inlet passage 7 (alternatively optional slave chamber inlet passage 17), slave chamber inlet orifice 15, lower region 21 of slave chamber 19, slave chamber outlet passage 27, slave chamber outlet orifice 29, upper region 35 of sensing chamber 31, sensing chamber outlet passage 43 and sensing chamber outlet orifice 45.

When in its closed position, the slave diaphragm 25 prevents a flow of oxygen, as the inhalation gas, out of the tube chamber 5 via outlet orifice 11 into the upper region 23 of slave chamber 19. When in its open position, the inhalation gas flows out of the upper region 23, into passage 47 and to an interior of the mask 39 via orifice 49. While FIG. 1 illustrates a single passage 51 fluidly communicating with passages 41, 47 in parallel, passage 51 is optional. In this alternative, both passage 41 and passage 47 separately fluidly communicate with an interior of the mask or nasal cannula 39 (hereinafter “mask”). Consistent with the foregoing, the skilled artisan will recognize that the inhalation gas flow path includes inhalation gas flow path inlet passage 9, inhalation gas flow path inlet orifice 11, upper region 23 of slave chamber 19, passage 47, orifice 49, passage 51, and mask 39.

In operation, when a person wearing the mask 39 inhales, the pressure difference across the sensing diaphragm 37 increases because a slight vacuum is induced in the lower region 33 of the sensing chamber 31. This increased pressure difference (P_{sensing upper}−P_{sensing lower}) is sufficient to overcome biasing of the sensing diaphragm 37 into its closed position by spring 53. Those of ordinary skill in the art will recognize that the biasing (P_{sensing break}) is a function of the configuration and material of construction of the sensing diaphragm 37 and of the bias applied by the spring 53. As a result, the timing gas (oxygen) is allowed to flow out of the slave chamber outlet passage 27, through the upper region 35 of the sensing chamber 31. The timing gas is thenceforth vented outside the conserver via sensing chamber outlet passage 43 and sensing chamber outlet orifice 45.

The initiation of the flow of timing gas through the timing gas flow path starts a timer whose expiration (after passage of predetermined time ΔT₁) initiates the flow of inhalation gas (oxygen) through the inhalation gas flow path. After passage of ΔT₁, a decrease in pressure in the lower region 21 of the slave chamber 19 increases the pressure differential (P_{slave upper}−P_{slave lower}) across the slave diaphragm 25 (i.e., pressure in upper region 23 versus pressure in lower region 21). This pressure differential increases because the flow of timing gas out of the conserver via the sensing chamber outlet passage 43 is relatively uninhibited while the flow of oxygen through the slave chamber inlet orifice 15 is inhibited due to its relatively small hydraulic diameter. As ΔT₁ expires, this increasing pressure differential exceeds the biasing of the slave diaphragm 25 towards its closed position. Those of ordinary skill in the art will understand that, due to the configuration and material of construction of the slave diaphragm 25, the biasing of the slave diaphragm towards its closed position can be characterized by a predetermined pressure differential (P_{slave break}). Thus, as ΔT₁ expires, the slave diaphragm 25 is caused to move to its open position.

When the slave diaphragm 25 moves to its open position, the flow of inhalation gas (oxygen) through the inhalation gas flow path is initiated and time ΔT₂ begins. During this time, the decrease in pressure in the upper region 35 of the sensing chamber 31 decreases a pressure differential across the sensing diaphragm 37 (P_{sensing upper}−P_{sensing lower}). As ΔT₂ expires, this decreasing pressure differential decreases to a point where it is longer sufficient to counter the biasing force from spring 53. As a result, the sensing diaphragm 37 moves back to its closed position and the sensing diaphragm 37 occludes the slave chamber outlet passage 27.

When the sensing diaphragm 37 moves to its closed position, time ΔT₃ beings, the flow of inhalation gas through the inhalation gas flow path continues, and the flow of timing gas through the timing gas flow path is prevented. During this time, the pressure in the lower region 21 of the slave chamber 19 start to increase. This causes the pressure differential (P_{slave upper}−P_{slave lower}) across the slave diaphragm 25 (i.e., pressure in upper region 23 versus pressure in lower region 21) to decrease. As ΔT₃ expires, this pressure differential is no longer sufficient to counter the biasing force of the slave diaphragm 25 and the slave diaphragm 25 moves to its closed position. This of course prevents the flow of inhalation gas through the inhalation gas flow path and delivery of the bolus of oxygen is completed

At any time after expiration of ΔT₃, the cycle of initiation of the timing gas flow, the initiation of the inhalation gas flow, the prevention of the timing gas flow, and the prevention of the inhalation gas flow may be repeated when the person wearing the mask 39 draws another breath. If a breath is drawn at the expiration of ΔT₄ or at some point in time after expiration of ΔT₄, the full bolus of oxygen intended to be delivered by the conserver will be delivered to the wearer. On the other hand, if a breath is drawn before passage of ΔT₄, although the pressure in the tubing reservoir may not have reached the regulated pressure provided by the oxygen source, a bolus of oxygen will still be delivered to the wearer. Nevertheless, this bolus will be smaller in comparison to the bolus delivered when a breath is drawn at or after expiration of ΔT₄ because it is prorated against the portion of ΔT₄ that passes before the breath is drawn.

As seen from the foregoing discussion, the conserver allows delivery of bolus of oxygen on demand by the wearer while at the same time preventing the delivery of more than a maximum minute volume flow rate of oxygen to the wearer. The minute volume rate delivered by the conserver may be expressed as:

[(bolus vol. delivered during ΔT₂ and ΔT₃)+(bolus vol. delivered during ΔX)]

(ΔT₁+ΔT₂+ΔT₃+ΔX).

where ΔX is the variable amount of time that passes after the flow of oxygen is prevented through the inhalation gas flow path and before a breath is subsequently drawn. Hence, ΔX depends upon when the wearer draws a breath. Imagine if the wearer draws breath precisely when the reservoir pressure reaches the regulated pressure, ΔX=ΔT₄ and the intended full bolus of oxygen is delivered. If the wearer instead draws breath after the reservoir pressure reaches the regulated pressure, ΔX>ΔT₄ and the same intended full bolus of oxygen is again delivered but over a longer period of time. If the wearer instead draws a breath before the reservoir pressure reaches the regulated pressure, ΔX<ΔT₄ and less than the intended full bolus is delivered because the reservoir pressure has not been “reset”. While these boluses are delivered to such a wearer more frequently in comparison to a wearer who waits until at least ΔT₄ passes and the denominator in the above equation is smaller, such boluses are also smaller because they are prorated according to the amount of time that passes before ΔT₄ expires.

Therefore, one can see that the wearer receives a same minute volume rate, regardless of how frequently breaths are drawn, so long as the wearer does not draw a breath before the reservoir pressure is reset. A constant minute volume rate is advantageous because it satisfies not only persons drawing breaths at a moderate rate but also persons taking smaller, more frequent breaths. The conserver also prevents the wastage of oxygen in the event that the wearer draws breaths less frequently than the sum of ΔT₁+ΔT₂+ΔT₃+A T₄.

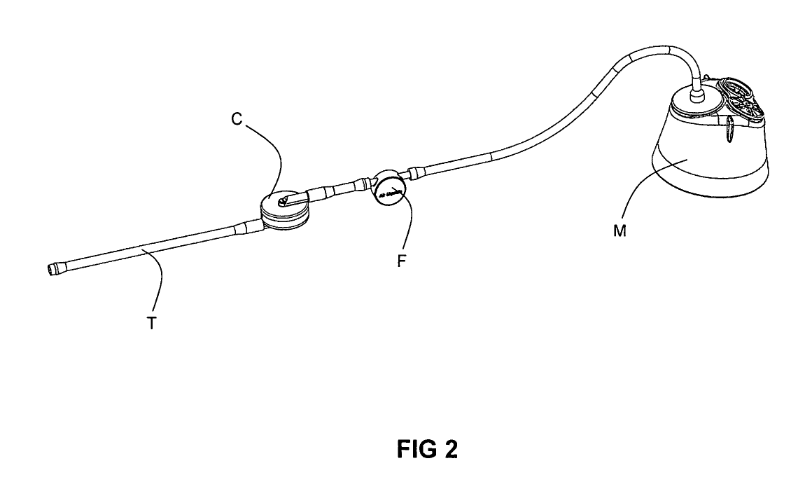

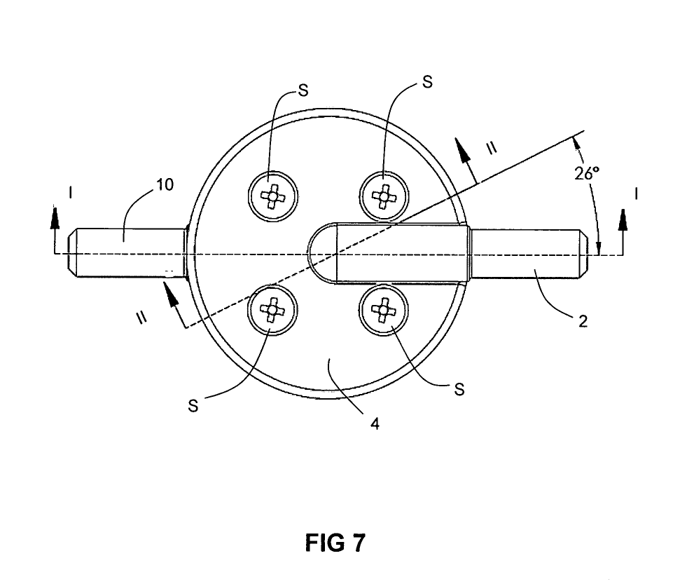

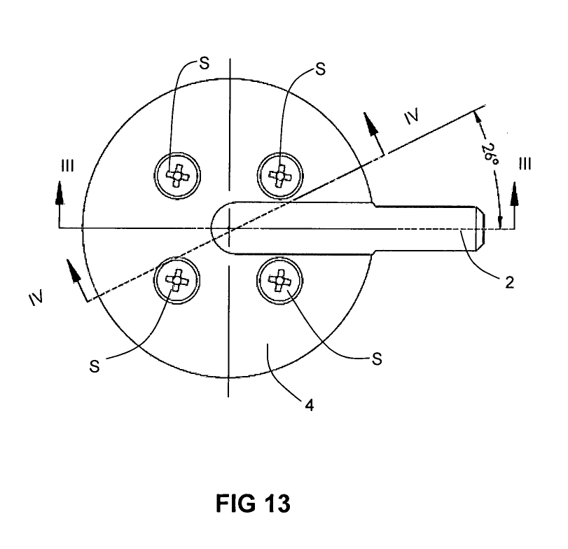

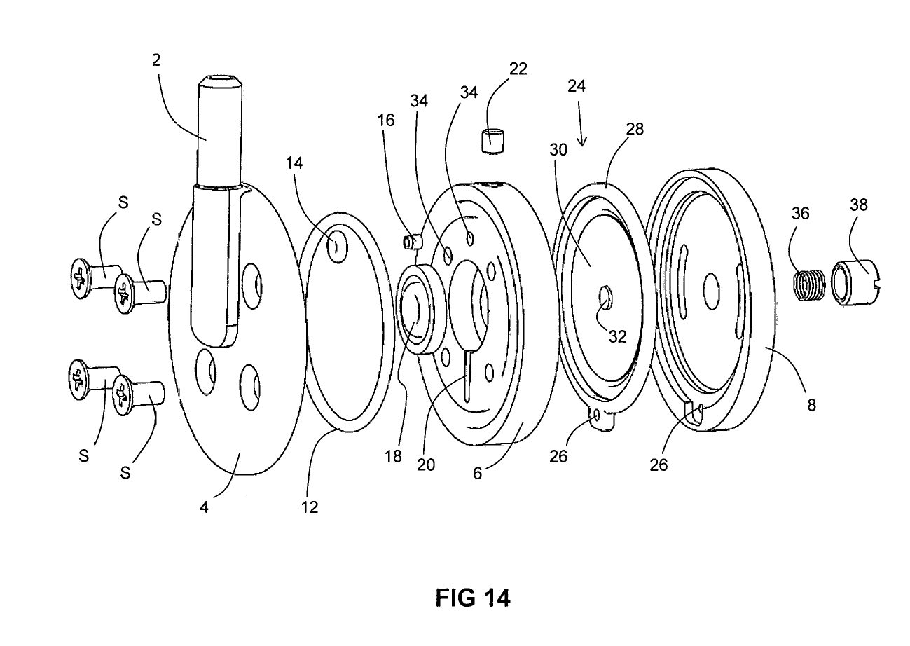

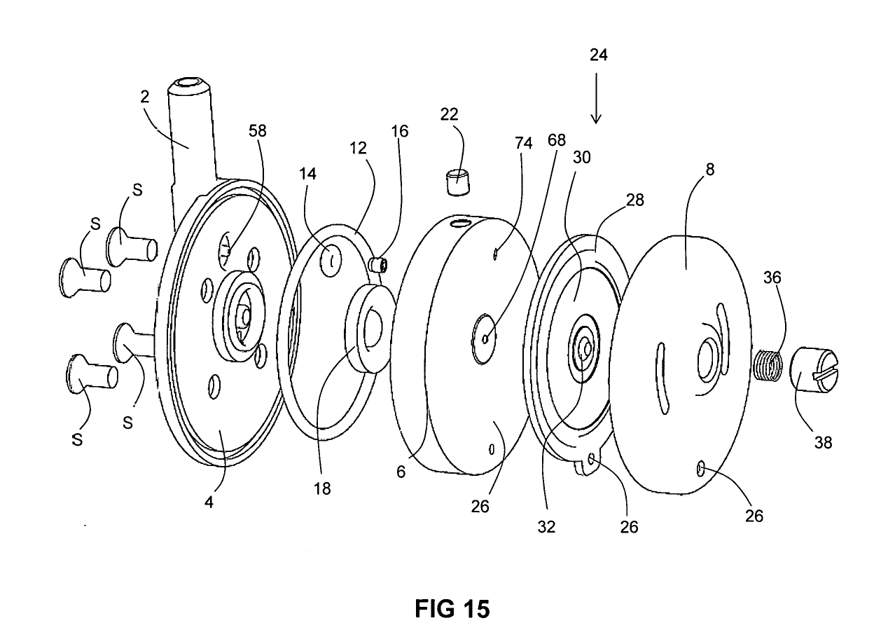

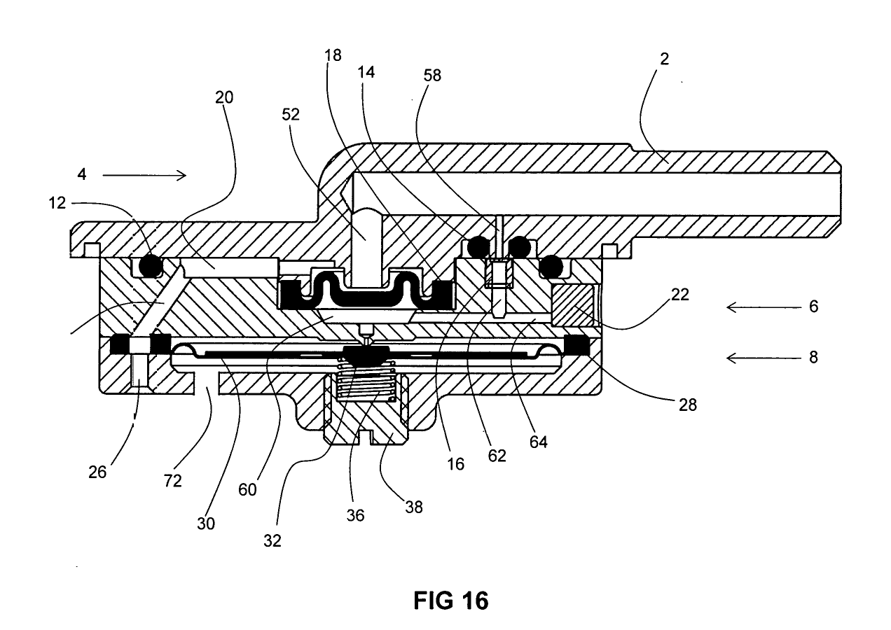

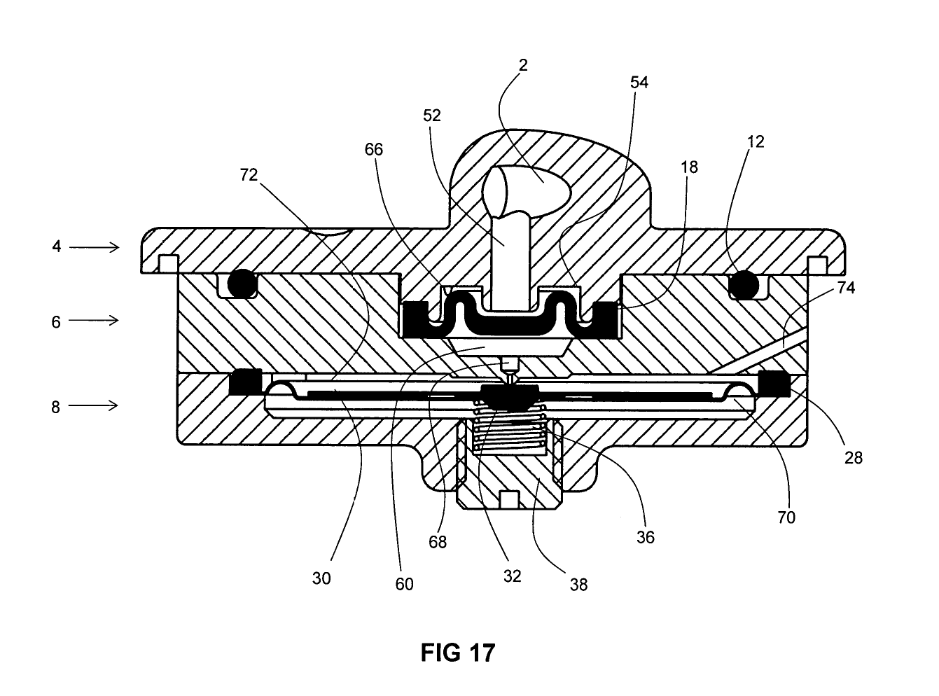

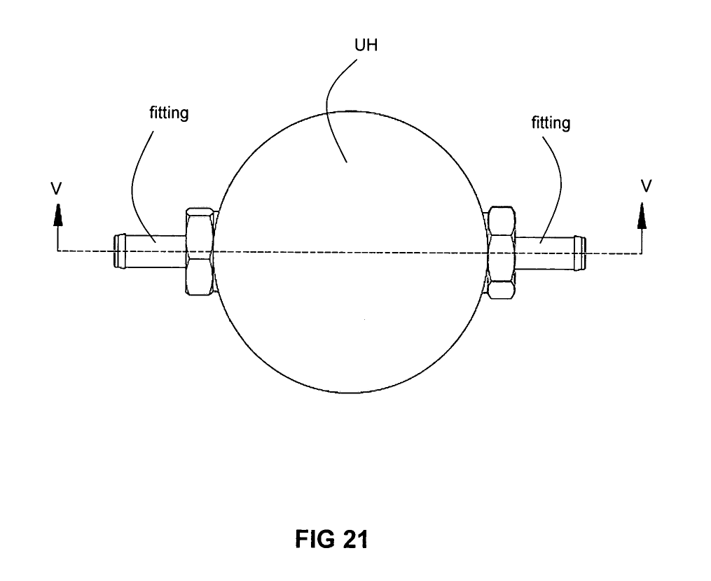

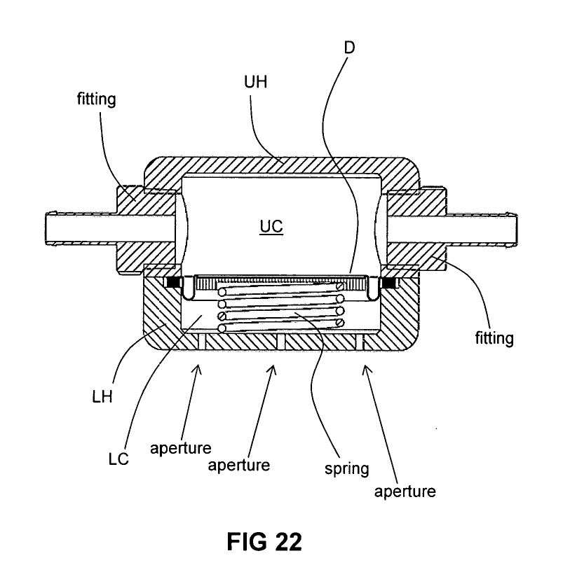

Particular embodiments of the inventive device will now be described with reference to FIGS. 2-22.

As best shown in FIG. 2, the oxygen delivery system includes tubing T, a conserver C, and an optional flow indicator F. In this instance, the conserver C delivers oxygen to a nasal mask M.

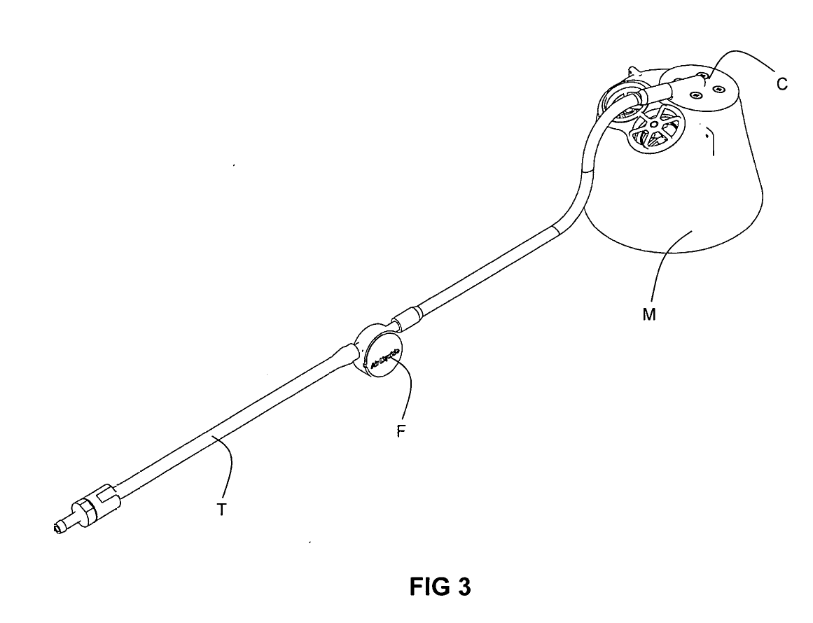

As best illustrated in FIG. 3 and in a different embodiment, the oxygen delivery system includes tubing T and a flow indicator F used in conjunction with a mask M. In contrast to the system of FIG. 2, however, the conserver C is integrated directly into the mask.

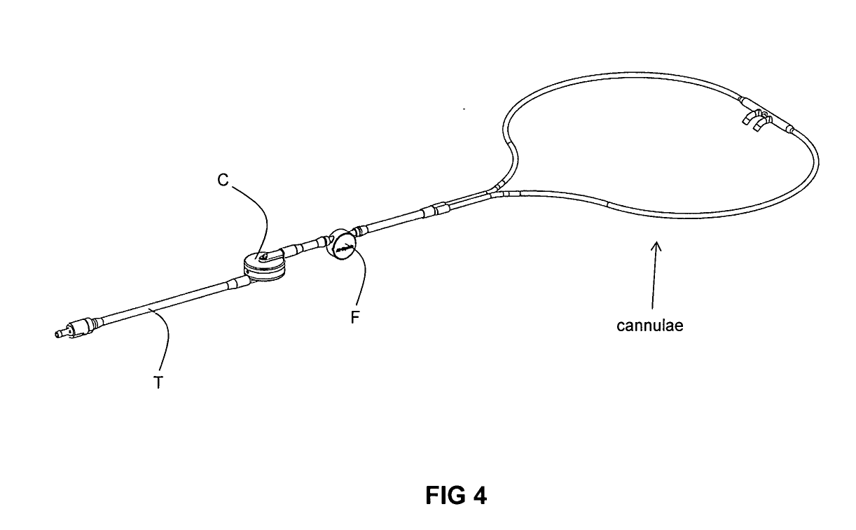

As best shown in FIG. 4 and in yet another embodiment, the oxygen delivery system includes tubing T, a conserver C, and a flow indicator F. In this instance, instead of feeding oxygen to a mask, it is fed to nasal cannula.

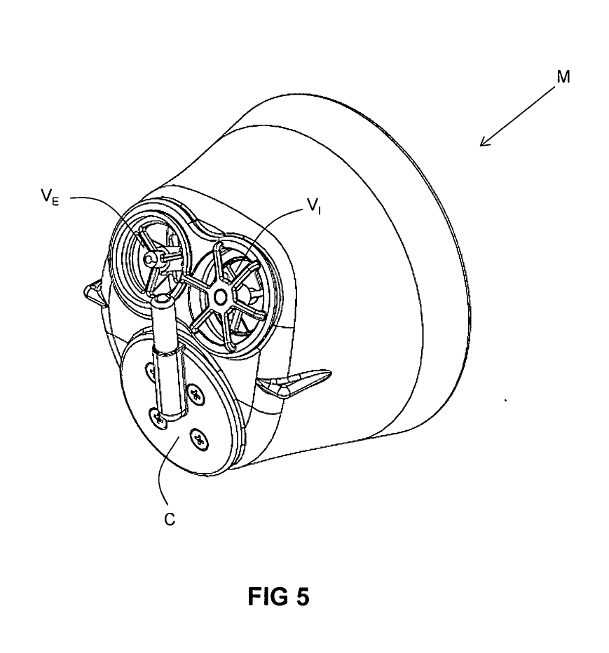

As best illustrated in FIG. 5, the integrated mask M includes an inspiratory valve V_{I} through which the mask wearer may draw in air and an expiratory valve V_{E} through which the mask wearer's exhalations may be vented from the mask M.

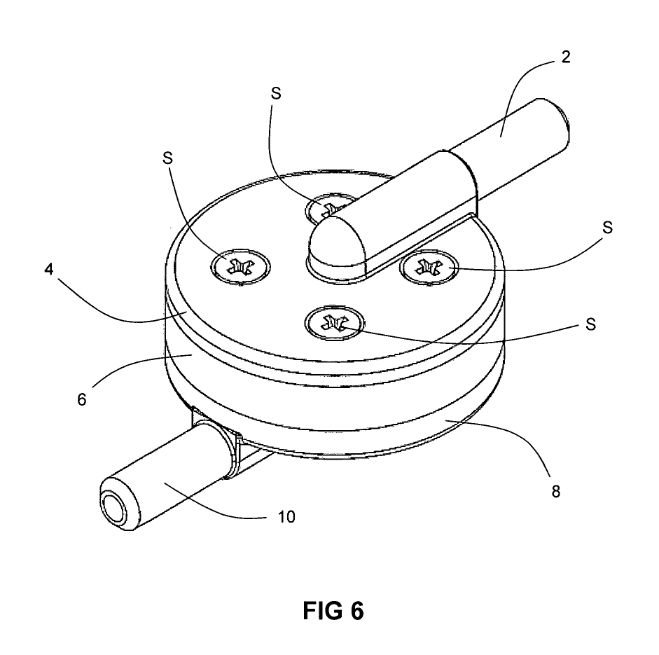

As best shown in FIGS. 6-11 and in the embodiment where the conserver is not integrated into a mask, the conserver includes an upper housing 4 and a lower housing 8 sandwiching a middle housing 6. At least the conserver housings 4, 6 may be secured to one another with a plurality of screws S. One of ordinary skill in the art will recognize, however, that the invention is not limited to such a technique. Ultrasonic welding and/or an adhesive may be used instead. The upper housing 4 includes a main body inlet 2 and the lower housing 8 includes a main body outlet 10.

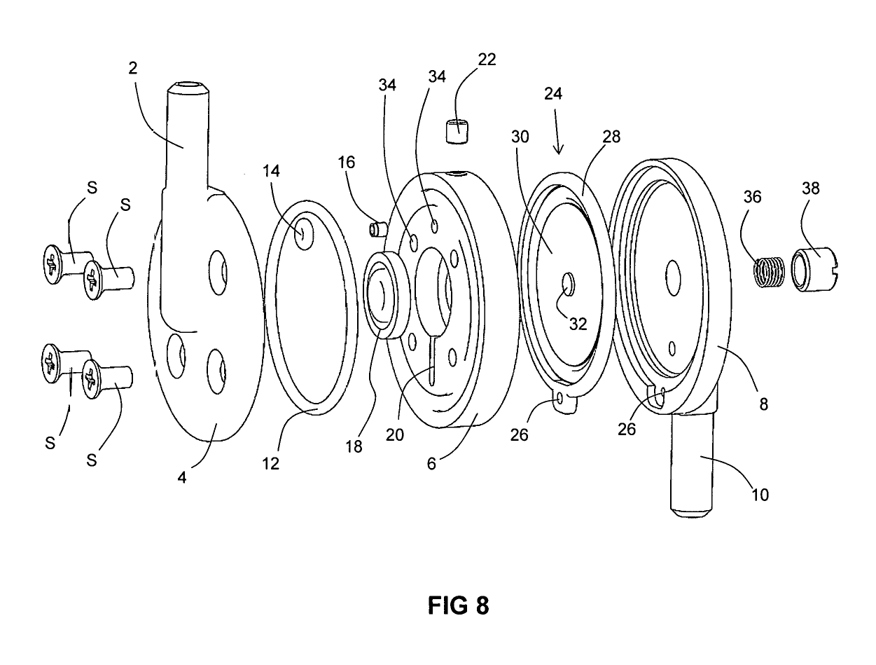

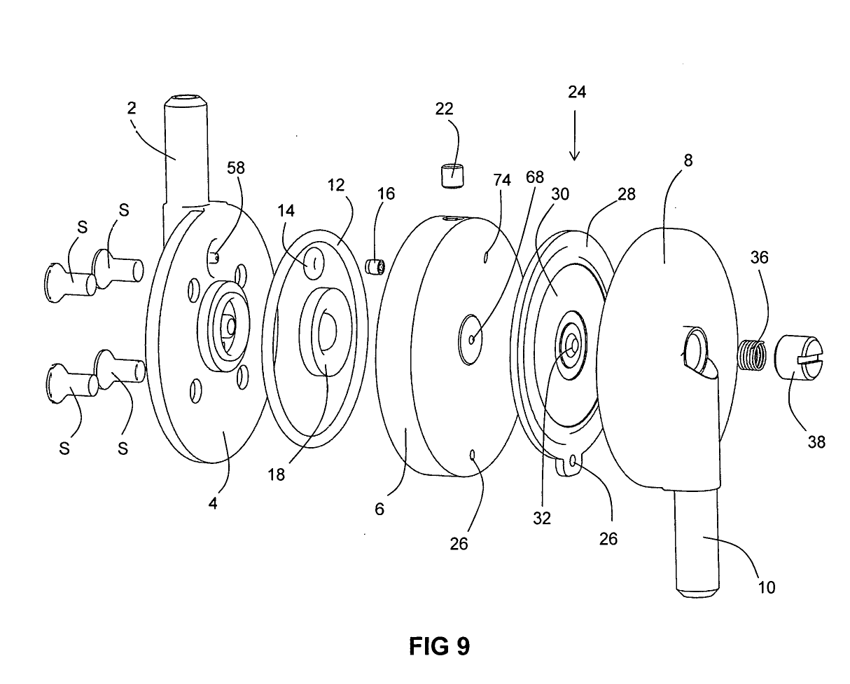

With reference to FIGS. 8-9, disposed in between the upper and middle housings 4, 6 are o-rings 12,14 and a slave diaphragm 18. A slave chamber inlet orifice 16 is inserted into any one of a plurality of cavities formed in the middle housing 6 that are adapted and configured to receive the orifice 16 in threaded connection or pressed interference fit. The o-ring 14 or similar seal serves to provide a gas-tight seal at the interface of the upper and middle conserver housings 4, 6 adjacent an upstream end of the orifice 16. The hollow passage of the orifice 16 is sized to provide a desired length of time during which the timing gas is allowed to flow through the timing gas flow path. A larger orifice 16 will allow a faster flow of the timing gas into the conserver and thus decrease ΔT₂ and ΔT₃. As a result, the volume of the bolus of gas released form the primary chamber tube 5 will be decreased. A smaller orifice 16 will of course have the opposite effect. Optionally, the conserver may be provided with a set of multiple o-rings or seals 14 and orifices 16 where the hollow passage of each orifice 16 has a different hydraulic diameter and each orifice 16 is received by an associated different cavity. In this manner, the conserver may be designed to deliver a variety of minute volume flow rates where the middle housing 6 need only be rotated in order to change the minute volume flow rate. Instead of, or in addition to, a set of multiple o-rings, seals 14, and orifices 16, one out of a variety of minute volume flow rates may be selected by providing a set of tubing inlet orifices having different hydraulic diameters.

Disposed in between the middle and lower housings 6, 8 is the sensing diaphragm 24. The sensing diaphragm 24 has dual functions. A thickened, peripheral portion 28 provides the function of an o-ring for providing a gas-tight seal between the conserver middle and lower housings 6, 8. A raised middle portion 32 serves to prevent a flow of timing gas through the timing gas flow path by occluding the slave chamber outlet orifice 68. An intermediate portion 30 connects the peripheral and middle portions 28, 32. A spring 36 provides the desired biasing of the sensing diaphragm 24 towards its closed position in which it occludes the slave chamber outlet orifice 68. The biasing force of the spring 36 may be adjusted by rotating screw cap 38. Screw cap 38 is threadedly received, in the manner of a nut and bolt, in a correspondingly sized cavity formed in the lower housing 8 to form a gas-tight seal and may be used to adjust the biasing force of the screw 36.

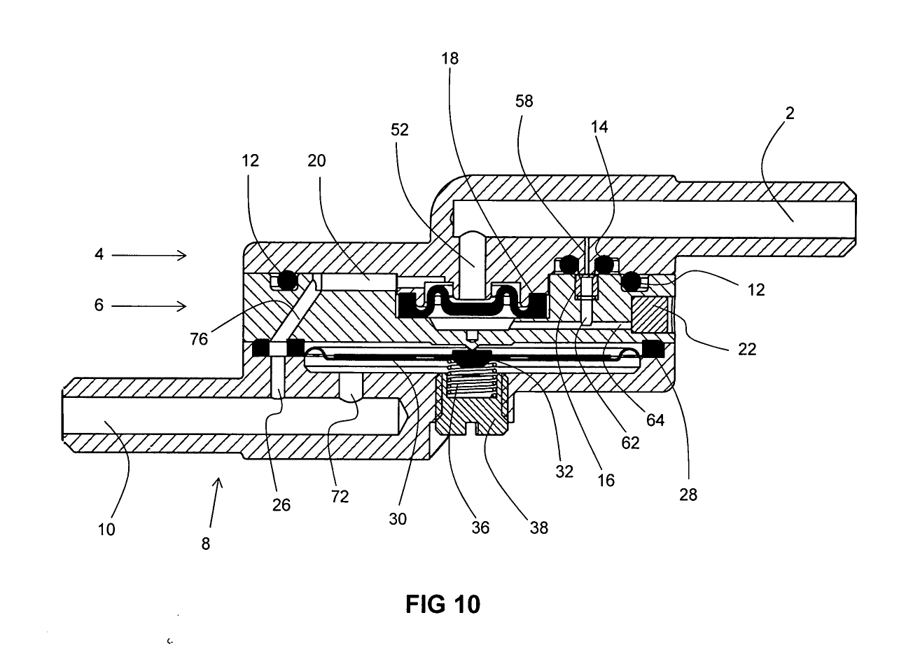

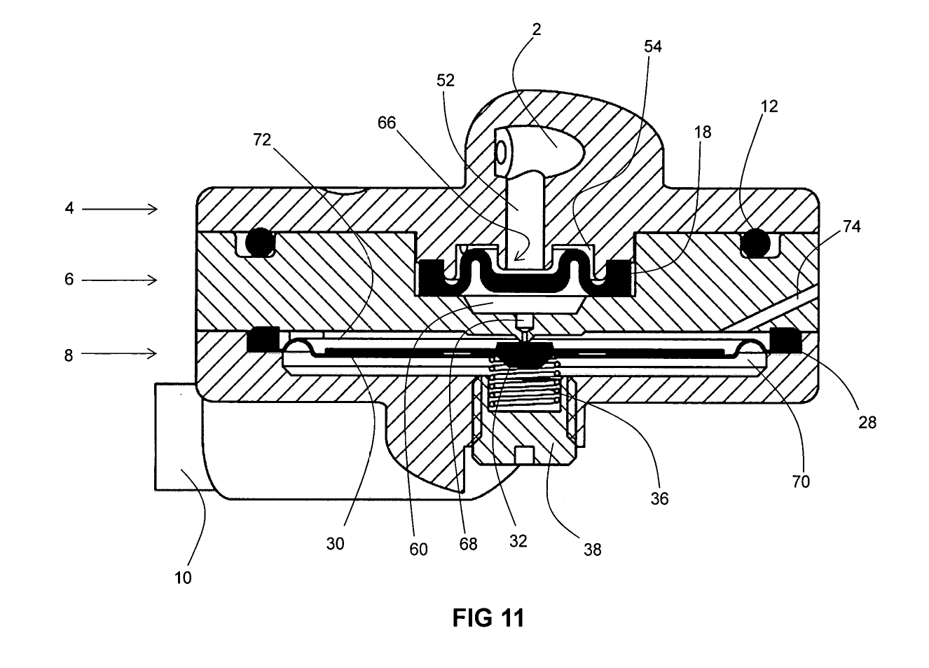

With reference to FIGS. 10-11, when the wearer of the mask or nasal cannula draws a breath, a slight vacuum induced in a lower region 70 of the sensing chamber (via main body outlet 10 and inhalation sensing passageway 72) causes the differential pressure across the sensing diaphragm 24 (P_{sensing upper}−P_{sensing lower}) to exceed the bias provided by spring 36 and move the sensing diaphragm 24 to its open position in which the middle portion 32 no longer occludes the slave chamber outlet orifice 68. As a result of the discontinuation of occlusion, oxygen through the timing gas flow path is no longer prevented and time period ΔT₁ begins.

With respect to the timing gas flow path, oxygen received from the tubing by main body inlet 2 is fed into an upstream end of a slave chamber inlet passage 58. When the sensing diaphragm is in its open position, the flow continues, in order, through timing gas inlet orifice 16, downstream end of slave chamber inlet passage 62, passage 64, a lower region 60 of the slave chamber, slave chamber outlet orifice 68, an upper region 72 of the sensing chamber, and is vented outside the conserver via vent orifice 74. This flow of the timing gas continues through time periods ΔT₁ and ΔT₂. During time period ΔT₁, the pressure within the timing gas flow path (including the lower region 60 of the slave chamber) decreases. At the expiration of time period ΔT₁, P_{slave upper}−P_{slave lower} exceeds P_{slave break} and the slave diaphragm 18 is moved to its open position and time period ΔT₂ begins.

With respect to the inhalation gas pathway, during time period ΔT₁, an abutment 66 formed on a surface of an upper region 54 of the slave chamber is in contact with the slave diaphragm 18 and prevents a flow of oxygen through the upper region 54. During time periods ΔT₁ and ΔT₂, oxygen is received from the tubing via inhalation gas inlet passage 52. The flow continues through an upper region 54 of the slave chamber and enters passages 20, 76, 26. Together, passages 20, 76, 26 comprise an inhalation gas outlet passage. Because the slave diaphragm 18 has been moved to its open position, the abutment 66 no longer prevents a flow of oxygen through the upper region. During ΔT₁, the pressure in the timing gas flow path decreases, however pressure increases in the upper region 72 of the sensing chamber.

At the expiration of ΔT₁, the pressure in upper region 72 of the sensing chamber has decreased to a point where P_{sensing upper}−P_{sensing lower} no longer exceeds the biasing force of spring 36 and the sensing diaphragm is moved to its closed position. As such, a flow of oxygen from the slave chamber outlet orifice 68 to the upper region 72 of the sensing chamber is prevented. Consequently, a flow through the timing gas flow path is prevented and the pressure thereof (including in the lower region 60 of the slave chamber) begins to build during time period ΔT₃.

At the expiration of ΔT₃, because of the building pressure in the lower region 60, P_{slave upper}−P_{slave lower} goes under P_{slave break}, the slave diaphragm 18 is moved to its closed position, and contact between abutment 66 and slave diaphragm 18 prevents a flow of oxygen through the upper region 54 of the slave chamber. After passage of ΔT₃, if the wearer of the mask or nasal cannula draws a breath, the cycle repeats with initiation of a flow of oxygen through the timing gas flow path and time period ΔT₁. During ΔT₄, the pressure in the tubing reservoir and the timing and inhalation gas flow paths builds back up to the regulated pressure provided by the oxygen source. Before ΔT₄ expires, any breath drawn by the wearer will result in the delivery of a bolus of oxygen that is prorated according to the degree of completion of ΔT₄.

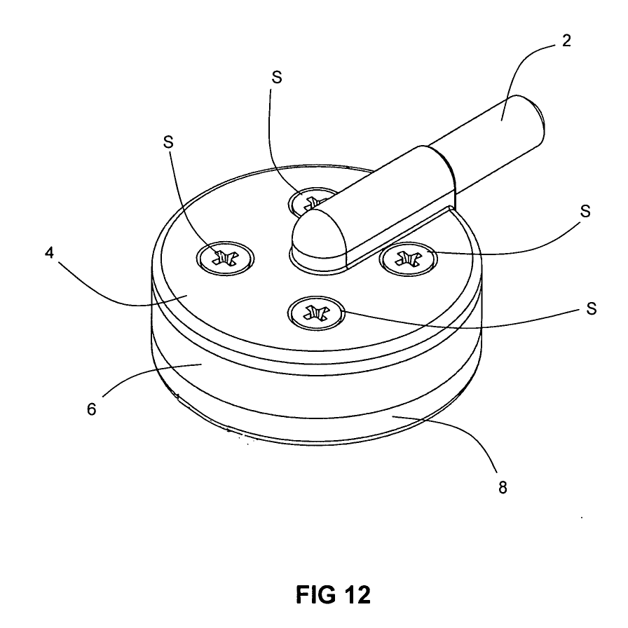

The conserver of the embodiment of FIGS. 12-17 is the same as that of FIGS. 6-11 with a few differences. The lower housing 8 is no longer provided with a main body outlet 10. This means that the slave chamber outlet passage made up of passages 20, 76, 26 and the inhalation gas sensing passageway emerge from the main body itself. This particular embodiment is useful when the conserver is integrated into a mask and thus does not need to be connected to any portion of tubing leading to the mask or nasal cannula.

With regard to the description of FIGS. 6-17 above, we note that, instead of the main body having a main body inlet, oxygen may be received by passages 52, 58 directly from the tubing.

In the context of an aeronautical vehicle based system, the invention may also include a device (i.e., an altitude adjustment device). In the case of cabin depressurization, as altitude is increased the ambient air pressure available for breathing in by the wearer via the inspiratory valve of the mask is decreased. Without adjusting for altitude, a conserver whose minute volume flow rate of oxygen is constant will fail to deliver enough oxygen to the wearer to compensate for the decreased amount of oxygen available from the subambient air. If such a constant minute volume flow rate conserver is sized so as to allow a minimum rate at very high altitudes, the conserver will deliver more than the needed rate at relatively lower altitudes and thus waste oxygen and unnecessarily increase the size and thus weight of the source of oxygen.

The altitude adjustment device of the invention acts to increase the minute volume flow rate of oxygen delivered by the conserver during an increase in altitude and decrease the minute volume flow rate of oxygen delivered by the conserver during a decrease in altitude. Any altitude adjustment known in the field of emergency oxygen supplies for aeronautical vehicles may be used in the invention.

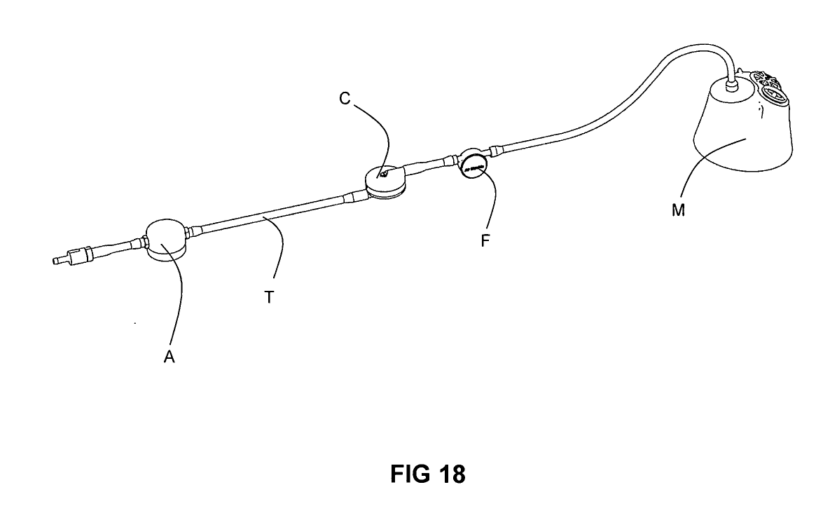

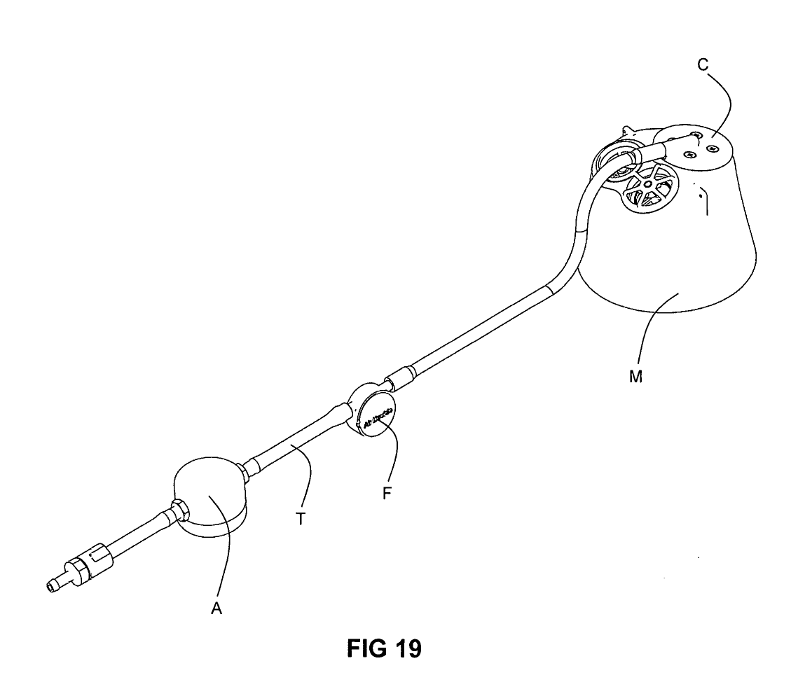

The embodiments of FIGS. 18 and 19 are the same as those of FIGS. 2 and 3, except that an altitude adjustment device A is disposed in-line with the tubing T downstream of any inlet orifice of the tubing T.

Proposed below are two altitude adjustment devices that adjust the minute volume flow rate of oxygen delivered by the conserver by mechanical means instead of with an electromechanical device.

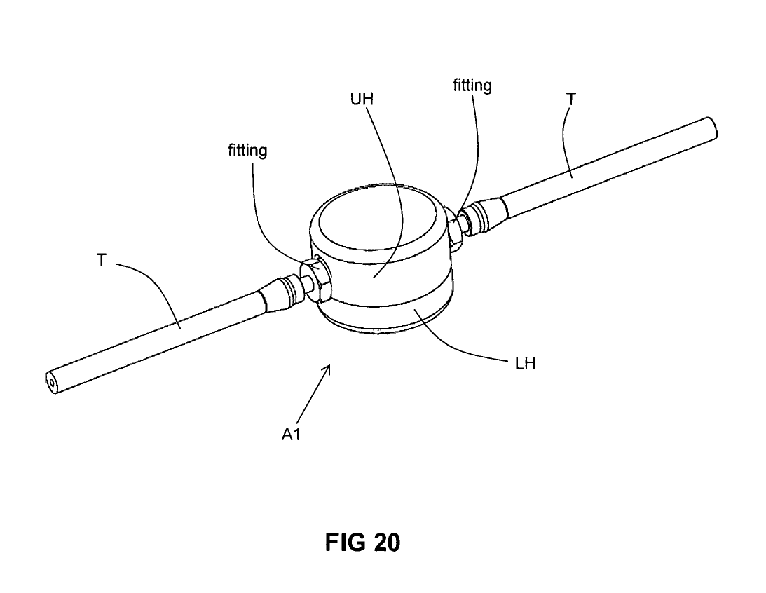

According to the first type of device, and as best shown in FIGS. 20-22, the altitude adjustment device A1 acts to increase a volume of the tubing reservoir downstream of the tubing inlet orifice during an altitude increase and decrease a volume of the reservoir during an altitude decrease. The altitude adjustment device A1 includes an upper housing UH secured to a lower housing LH in gas-tight fashion. Upstream and downstream ends of the device A1 are connected in gas-tight fashion to upstream and downstream portions of the tubing T via fittings. The interior of the device A1 is divided into upper and lower cavities UC, LC by a diaphragm D. The diaphragm is biased towards the upper cavity UC by a spring. While the upper cavity UC is in fluid communication with the fittings, the lower cavity LC is not. The lower cavity LC is also not gas-tight in that a plurality of apertures are formed in the lower housing so that the pressure is equalized between the lower cavity LC and the ambient atmosphere outside the device. During an increase in altitude, the ambient pressure decreases while the pressure inside the upper cavity UC remains the same. As a result, the diaphragm D moves downwardly to expand the volume of the upper cavity UC. In this manner, the volume of the tubing reservoir is increased so as to increase the minute volume flow rate delivered by the conserver. Conversely, during a decrease in altitude, the ambient pressure increases while the pressure inside the upper cavity UC remains the same. As a result, the diaphragm moves upwardly to decrease the volume of the upper cavity UC. In this manner, the volume of the tubing reservoir is decreased so as to decrease the minute flow volume rate delivered by the conserver.

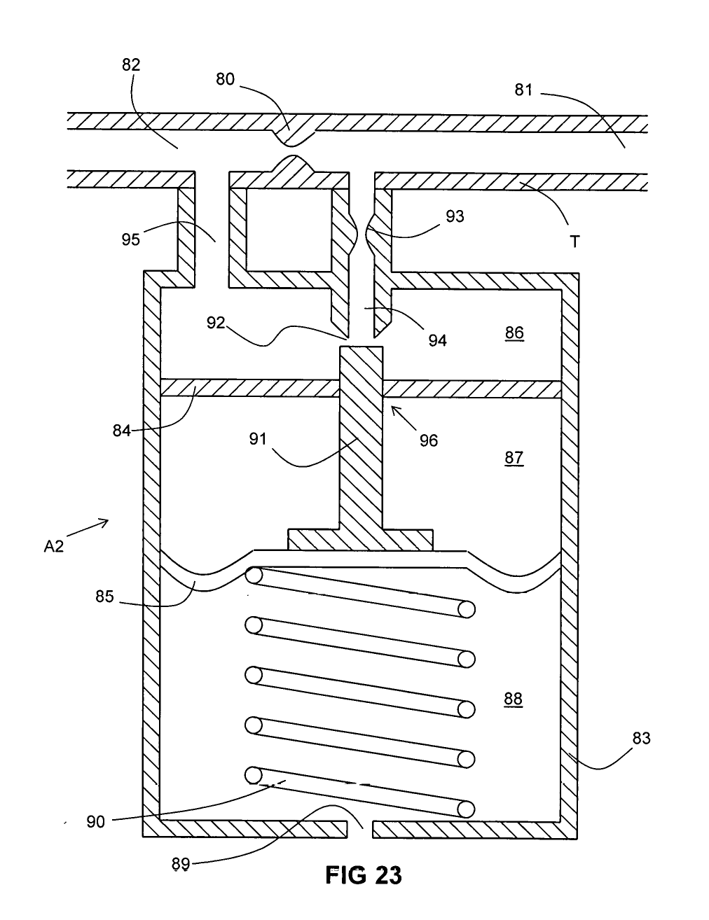

According to the second type of device, and as best shown in FIG. 23, the altitude adjustment device A2 acts to increase the amount of oxygen passed through the tubing inlet orifice during an altitude increase and decrease that amount during an altitude decrease. The altitude adjustment device A2 is secured to the tubing T adjacent a tubing inlet orifice 80. A interior of the altitude adjustment device A2 enclosed by a housing 83 is divided by a rigid wall 84 and a flexible diaphragm 85 into an upper region 86, a middle region 87, and a lower region 88. The lower region is pressure-equalized to ambient by way of opening 89. The middle region 87 is gas-tight and filled with a gas at atmospheric pressure. The upper region 86 is also gas-tight (but for fluid communication with tubing T) and pressure-equalized with the pressure in the tubing T.

Although FIG. 23 depicts the altitude adjustment device A2 in its open position, a spring 90 has a biasing force designed to push a rod 91 upward so as to occlude a valve seat 92 when the altitude adjustment device A2 is at ground level so as to prevent oxygen from an upstream portion 81 of tubing T from flowing through the altitude adjustment device inlet 94 and into upper region 86.

During an increase in altitude, the pressure difference across the diaphragm 85 increases because, while the middle region 87 is gas-tight and filled with a gas at atmospheric pressure, the lower region 88 is at subambient due to the pressure equalization afforded by opening 89. The biasing force of the spring 90 is set so that, above a predetermined altitude, the pressure difference across the diaphragm 85 is sufficient to slightly compress the spring 90 and lower the rod 91. As a result, the valve seat 92 is no longer occluded by the rod 91 and oxygen is allowed to flow from an upstream portion 81 of the tubing T to a downstream portion 82 of tubing T via inlet 94, secondary orifice 93, upper region 86, and altitude adjustment device outlet 95. As the gap between the open end of the inlet 94 and the opposing face of the rod 91 increases, the resistance to oxygen flow decreases. The presence of the secondary orifice 93 prevents the oxygen from bypassing the orifice 80. In this manner, a greater amount of oxygen is allowed to flow into the tubing reservoir downstream of inlet 80. The minute volume flow rate of oxygen delivered by the conserver is increased, during ΔT₂ and ΔT₃, the pressure in the upper region of the slave chamber will decrease more slowly in comparison to the case where a flow of oxygen is not permitted past orifice 93. Because the pressure decreases more slowly, a greater amount of oxygen flows through the inhalation gas pathway of the conserver.

As an alternative to the two above-described altitude adjustment devices A1, A2 shown in FIGS. 20-23, other mechanical structures adapted and configured to perform the same function may be used. For example, a portion of the tubing may be made of a material having a relatively lower hardness (thus having an inherently greater flexibility) than other portions of the tubing) that expands during an increase in altitude and contracts during a decrease in altitude. As one more example, instead of a spring-biased diaphragm, a stand-alone rolling diaphragm or a piston may be used.

While the inventive system may be used for anything requiring a controlled flow of gas delivered in boluses in a cyclical fashion, the inventive gas demand device is typically used by either a patient in gas therapy, such as oxygen therapy with oxygen, oxygen-enriched gas, or compressed air, or by the crew or passengers of an aircraft during low oxygen and/or pressure conditions.

Whether used by a patient for gas therapy or by aircraft crew in low oxygen and/or low pressure environments, in comparison to conventional gas demand devices, the inventive device has several advantages.

The inventive device reduces the required size and/or weight of an oxygen supply vessel (such as a compressed gas cylinder) and/or increases the time of use in between successive refilling or replacement of the vessel. Decreased size and/or weight are important in the gas therapy context for patients who may experience, muscular weakness, lack of muscle tone, and/or lack of stamina. Decreased size and/or weight will also ordinarily result in decreased costs for the manufacturer, insurer, and/or patient. In contrast to the demand device disclosed in U.S. patent application Ser. No. 15/255,858, filed Sep. 2, 2016, there is no need to provide a primary chamber (comparable to the tubing reservoir of the instant invention) within the main body. Because the tubing reservoir serves the function of the primary chamber of the device of the '858 application, the volume encompassed by the main body of the instant invention need not be increased to accommodate the volume of such a primary chamber. Because the volume of the main body of the instant invention need not be so large, the material of construction of the main body need not be so heavy.

Decreased size and/or weight are also important in the aerospace context. Aerospace oxygen systems are typically only used in the rare occurrence of a cabin depressurization in the worst case or to provide first aid to an ill passenger. Regardless, every flight must care enough oxygen supply to meet the worst case scenario. Consequently, the weight of these systems on board consumes fuel, reduces payload and range and increases operating costs. Current systems for on-board oxygen provide continuous flow oxygen. Continuous flow limits the duration of time during which the oxygen is supplied. Continuous flow also requires the maximum size supply vessel that the storage space. When an aircraft is used with a same continuous flow system on both short-distance and long-distance flights, the oxygen requirements for the long-distance flight will control. Thus, long-distance flights with a bulkier and/or heavier continuous flow system that decreases fuel consumption. While short-distance flights may utilize a less bulky and/or less heavy continuous flow system, such a system will limit the range of the aircraft on a subsequent flight unless the system is swapped out with a more bulky and/or heavier continuous flow system. By using the inventive gas demand device, the weight can be reduced. Therefore, the aircraft range and/or payload may be increased and fuel consumption decreased. Indeed, in comparison to some conventional systems, the inventive device can reduce the amount of oxygen required on the typical aircraft by as much as 75%. The weight reduction achievable by the inventive device can also improve safety and maintenance costs as well enabling the use of compressed gas cylinders rather than chemical oxygen generators.

The conserver has also been designed to be made of fewer components and lightweight materials, such as plastic and typically high density polyethylene. This simplifies the conserver resulting in better reliability and ease of manufacture. It will also reduce the conserver's weight which will save fuel when utilized in aerospace. This lighter weight can also help maintain a good mask seal on the wearer's face. If a conserver is too heavy (such as conventional conservers), it can pull the mask down and create a leak between the user's face and the mask, thereby causing the inhalation sensing feature of the conserver to fail and not be triggered by the wearer's inspiratory breath. Many conventional devices are electrically powered with a battery and may suffer from power failures, voltage errors and are generally heavier due to the weight of the battery. In contrast, the inventive device functions pneumatically and does not require any electrical power or batteries.

The conserver is designed to be sensitive enough to trigger at a negative pressure less than the cracking pressure of the masks inspiratory valve but less sensitive then similar devices that could cause false triggering if used on a mask. However the design when used with a nasal cannula has good sensitivity due to its close proximity to the user's end of the cannula as opposed to most systems where the conserver is integrated into a regulator on an oxygen cylinder and distant from the user, often 7 feet.

Many conventional devices include features which are freely movable within the device and which may be impacted by the relative position of the device by the force of gravity. For example, some conventional devices may include a ball-type check valve intended to reduce the amount of back pressure created when a pulse of oxygen exits the device and prematurely forces a diaphragm closed. This type of valve is a positional valve that only functions properly when the device is in an orientation where gravity keeps the check valve ball away from its seat. Should the device be inverted the check valve ball will fall to its seat and occlude the passage to the diaphragm, the device may not function since the check valve ball can remain to occluded if the inspiration from the user is not great enough to lift the ball from its seat. The amount of negative pressure (<−1.00 cm H₂O) typically created by the user at an outlet of such device would most likely not be enough to lift the check valve ball off its seat. In contrast, operation of the inventive device does not depend upon how it is positioned or oriented. In other words, the main diaphragm will not be prematurely closed and operation will not change if the position of the inventive device is changed.

The inventive design is small and easy to use and can be configured and adapted to several modalities such as compressed gas high pressure cylinders, cryogenic oxygen systems, oxygen generators, institution wall oxygen gas outlets, portable oxygen systems and remotely piped oxygen systems.

While some conventional home healthcare gas demand systems have been tried in the commercial aviation market with limited acceptance and success, most were not durable enough for the rigors of the commercial aviation market or provide inadequate interface with the aircraft storage system. The inventive device has been designed with the commercial aviation market in mind in order to overcome the problems experienced by many conventional systems as well as for the healthcare market so as to improve upon current gas demand systems for the healthcare market.

Conventional continuous flow compressed gas systems have a limited use time (for inhalation by the user) that is based upon the volume and pressure of the gas cylinder. Put quite simply, the use time is determined by dividing the mass of gas in the cylinder by the flow rate. In contrast, for the same mass of gas in the gas cylinder, the inventive device extends the use time (for inhalation by the user) because it does not use a continuous flow.

Many conventional gas demand devices tend to be complicated, do not control the volume of gas delivered over time, and do not provide the desired pulse bolus flow curve (i.e., a relatively high peak flow for a short duration) that is best for the person using the device. On the other hand, the inventive device provides the desired bolus flow curve.

Some conventional devices deliver multiple pulses in rapid succession creating a saw tooth gas flow pattern that is depend on constant inhalation and does not control the flow over time. In contrast, the inventive device supplies a bolus of gas upon user demand (i.e., inhalation by the user). Thus, it does not deliver another bolus of gas unless it is demanded by the user.

In comparison to many conventional devices, the inventive device exhibits increased reliability, performance, and ease of use, and a decreased rate of failure caused by uncontrolled user interfaces and real world user conditions.

Most conventional devices depend upon either a back pressure from the gas delivery line or back pressure at an outlet of the device in order to close a main diaphragm and reset its pneumatic circuit. The dependence of a back pressure for closing the main diaphragm is because the last orifice upstream of the outlet is located downstream of a fluidic passage to the diaphragm in question. This particular arrangement will result in a varying back pressure upon the diaphragm; consequently, cause an inconsistent volume per minute delivery.

In contrast, the internal features of the tubing and conserver work together create a timing circuit that is independent of any back pressure exerted onto the sense diaphragm. The sense diaphragm of the inventive conserver resets itself based upon the pneumatic timing circuit and the bias of the spring. Upon movement of the sensing diaphragm to its open position, a “secondary slave chamber” in the upper region of the sensing chamber is created. This secondary slave chamber adds to the timing circuit to ensure the main diaphragm followed by the slave diaphragm does not close before the tubing reservoir volume and pressure is depleted. This is important to ensure any back pressure on the sense diaphragm does not affect the timing and the minute volume is consistent across typical range of breath rates.

An ongoing challenge for most conventional pneumatic demand devices is the ability to be sensitive enough for the person with slow shallow breaths to trigger the device without the device being over sensitive to variations in the gas inlet pressure resulting in the device to self-cycle (auto-pulse). Overly complicated designs exacerbate this sensitivity problem since they magnify the amplitude of any pressure deviations from the specified regulated pressure.

In contrast, the design of the pneumatic circuit of the inventive conserver is simplified, so the amplification of pressure-sensitivity experienced by many conventional devices is significantly dampened in the inventive device. The simplified design of the pneumatic circuit also increases the ease of manufacturing, reduces component count and improves performance. To put a finer point on this assertion, the geometry of the components that make the slave chamber and sensing chamber and the orifices formed in the main body of the conserver are designed to reduce the quantity of components and the cost of the components for manufacturing the device. For example, the device of U.S. Pat. No. 7,089,938 may use as many as 22 components making up the pneumatic circuit while the inventive device may use as few as 15 components.

Legends

| A | first altitude adjustment device |
|---|---|
| C | conserver |
| D | diaphragm for first altitude adjustment device |
| F | flow indicator |
| LC | lower cavity |
| LH | lower housing of altitude adjustment device |
| M | mask |
| S | screw |
| T | tubing |
| UC | upper cavity |
| UH | upper housing of altitude adjustment device |
| V_{E} | expiratory valve |
| V_{I} | inspiratory valve |
| 1 | tubing orifice |
| 2 | main body inlet |
| 3 | tubing inlet |
| 4 | upper conserver housing |
| 5 | tubing downstream of tubing orifice |
| 6 | middle conserver housing |
| 7 | slave chamber inlet passage |
| 8 | lower conserver housing |
| 9 | inhalation gas flow path inlet passage |
| 10 | main body outlet |
| 11 | inhalation gas flow path inlet orifice |
| 12 | o-ring |
| 14 | o-ring |
| 15 | slave chamber inlet orifice |
| 16 | slave chamber inlet orifice |
| 17 | optional slave chamber inlet passage |
| 18 | slave diaphragm |
| 19 | slave chamber |
| 20 | Inhalation gas passage |
| 21 | lower region of slave diaphragm |
| 22 | plug |
| 23 | upper region of slave diaphragm |
| 24 | sense diaphragm |
| 25 | slave diaphragm |
| 26 | inhalation gas passage |
| 27 | slave chamber outlet passage |
| 28 | peripheral portion of sensing diaphragm |
| 29 | slave chamber outlet orifice |
| 30 | middle portion of sense diaphragm |
| 31 | sense chamber |
| 32 | raised portion of sense diaphragm |
| 33 | lower region of sense chamber |
| 35 | upper region of sense chamber |
| 36 | spring |
| 37 | sense diaphragm |
| 38 | screw cap |
| 30 | mask |
| 41 | inhalation sense Passage |
| 43 | sense chamber outlet passage |
| 45 | upper region of sense chamber outlet orifice |
| 47 | inhalation gas passage |
| 51 | inhalation gas and sense passage |
| 52 | inhalation gas inlet passage |
| 53 | spring |
| 58 | upstream end of slave chamber inlet orifice |
| 60 | lower region of the slave chamber |
| 62 | downstream end of slave chamber inlet orifice |
| 64 | passage |
| 66 | inhalation gas flow path inlet orifice |
| 68 | slave chamber outlet orifice |
| 70 | lower region of sensing chamber |
| 72 | inhalation sense passageway |
| 74 | upper region of sense chamber outlet orifice |
| 76 | inhilation gas passage |

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within the range.

All references identified herein are each hereby incorporated by reference into this application in their entireties, as well as for the specific information for which each is cited.

What is claimed is:

1. A pneumatic oxygen conserving system for delivering oxygen for inhalation, comprising tubing that is adapted and configured to receive gas from a source of gas, a conserver in downstream flow communication with said tubing, and a wearable gas distribution device comprising a face mask, a nasal mask, or nasal cannula in downstream flow communication with said conserver that is adapted and configured to deliver oxygen to a person for inhalation thereof, the conserver comprising a main body into which is formed: a vent orifice that opens out of the main body; a slave chamber divided into upper and lower regions by a slave diaphragm; a sensing chamber divided into upper and lower regions by a sensing diaphragm; an inhalation sensing passageway that opens into the main body in upstream flow communication with the sensing chamber lower region; a slave chamber inlet passage fluidly communicating between the main body inlet and the slave chamber upper region; a slave chamber outlet passage in flow communication between the slave chamber upper region and the mask or nasal cannula; a timing gas inlet passage in upstream fluid communication with the slave chamber lower region; and a timing gas outlet passage fluidly communicating between the slave chamber lower region, the sensing chamber upper region, and the vent orifice, wherein:
an inhalation gas flow path is comprised of said slave chamber inlet passage, said slave chamber upper region, and said slave chamber outlet passage;
a timing gas flow path is comprised of said timing gas inlet passage, said slave chamber lower region, said timing gas outlet passage, and said vent orifice;
the slave diaphragm is biased to a closed position in which it occludes flow communication between the slave chamber inlet and outlet passages and a flow of oxygen through the inhalation gas flow path is prevented;
the slave diaphragm is moved from its closed position to its open position when a difference in pressure, P_{slave upper}−P_{slave lower}, between the slave chamber upper and lower regions, respectively, exceeds a predetermined differential pressure P_{slave break};
the sensing diaphragm is biased in a closed position in which it occludes flow communication between timing gas inlet and outlet passages and prevents a flow of oxygen through the timing gas flow path;
the sensing diaphragm is moved from its closed position to its open position when a difference in pressure, P_{sensing upper}−P_{sensing lower}, between the secondary slave chamber upper and lower regions, respectively, exceeds a predetermined differential pressure P_{sensing break}; and
the conserver is adapted and configured such that:
when the sensing and slave diaphragms are in their closed positions, inhalation of a person wearing the mask or nasal cannula will cause P_{sensing upper}−P_{sensing lower} to exceed P_{sensing break} and move the sensing diaphragm to its open position, oxygen flows out of the vent orifice via the main body inlet and the timing gas inlet and outlet passages, and an oxygen flow out of the inhalation gas outlet passage is delayed by a time period ΔT₁;
at the expiration of ΔT₁, a decrease in pressure in the timing gas flow path causes P_{slave upper}−P_{slave lower} to exceed P_{slave break} and the slave diaphragm is moved to its open position and oxygen flows through the inhalation gas flow path while the sensing diaphragms remains in its open position for a time period ΔT₂;
at the expiration of ΔT₂, P_{sensing upper}−P_{sensing lower} drops below P_{sensing break} and the sensing diaphragm is moved to its closed position and oxygen is prevented from flowing through the timing gas flow path while oxygen continues to flow through the inhalation gas flow path for a time period ΔT₃;
at the expiration of ΔT₃, an increase in pressure in the timing gas flow path causes P_{slave upper}−P_{slave lower} to drop below P_{slave break} and the slave diaphragm is moved to its closed position and flows of oxygen are prevented through the timing and inhalation gas flow pathways; and
during ΔT₂ and ΔT₃, a bolus of oxygen delivered by the conserver to the mask or nasal cannula is drawn from a reservoir in the tubing.

2. The oxygen delivery system of claim 1, wherein the conserver further comprises a main inlet in flow communication between the tubing and the inhalation and timing gas inlet passages so as to receive flows of oxygen via the tubing and the main body inlet.

3. The oxygen delivery system of claim 1, wherein the conserver further comprises a main inlet in flow communication between the tubing and the inhalation and timing gas inlet passages and the timing gas inlet passage is in downstream flow communication with the tubing so as to receive a flow of oxygen via the tubing but not from the main body inlet.

4. The oxygen delivery system of claim 1, wherein the wearable gas distribution device is a face mask or nasal mask comprising an inspiratory valve and an expiratory valve, the conserver is integrated into the face mask or nasal mask, the inhalation and timing gas inlet passages are in fluid communication with a downstream end of the tubing, and the inhalation gas outlet passage opens out into an interior of the face mask or nasal mask.

5. The oxygen delivery system of claim 1, wherein the wearable gas distribution device is nasal cannula and the conserver is disposed in-line with the tubing.

6. The oxygen conserving system wherein the conserver is made of a plastic material.

7. The oxygen conserving system, further comprising an altitude adjustment device disposed downstream of the tubing orifice in-line with the tubing that is adapted and configured to keep the predetermined minute volume flow rate constant when a decrease or increase in ambient pressure caused by an increase or decrease in altitude, respectively.

8. An oxygen delivery system comprising an oxygen source, a pressure regulator, and the pneumatic oxygen conserving system of claim 1, wherein the pressure regulator is adapted and configured to regulate a pressure of the oxygen source to a lower pressure for delivery into the tubing, the tubing includes an orifice choking a flow of oxygen therethrough that separates the tubing into upstream and downstream portions, the one or both of the inhalation gas inlet and outlet passages includes an orifice choking a flow of oxygen therethrough, and the downstream portion of tubing is sized and the orifice(s) of the inhalation gas inlet and outlet passages are dimensioned so as to achieve a predetermined minute volume flow rate of oxygen to the wearable gas distribution device.

9. The oxygen delivery system of claim 8, further comprising an altitude adjustment device disposed downstream of the tubing orifice in-line with the tubing that is adapted and configured to increase or decrease a minute volume flow rate delivered by the conserver when a decrease or increase in ambient pressure, respectively, is caused by an increase or decrease in altitude, respectively.

10. The oxygen delivery system of claim 9, wherein the altitude adjustment device comprises a housing having an inlet, an outlet, at least one vent holes on an end thereof that opens out to an exterior of the housing and the ambient atmosphere, and an altitude adjustment diaphragm dividing an interior of the housing into first and second regions, the first region being in flow communication with the housing inlet and outlet, the second region being in flow communication with each of the one or more vent holes, the altitude adjustment diaphragm being biased with a spring into a rest position, a decrease in ambient pressure caused by an increase in altitude causing the diaphragm to move against the biasing of the spring, decrease the volume of the second region, and increase the volume of the first region and consequently increase the volume of the tubing downstream portion.

11. The oxygen delivery system of claim 9, wherein the altitude adjustment device comprises a section of the tubing having a flexibility that is greater than remaining sections of the tubing so that a decrease in ambient pressure caused by an increase in altitude causing the section of the tubing comprising the altitude adjustment device to expand outward and increase the volume of the tubing downstream portion

12. The oxygen delivery system of claim 8, wherein the altitude adjustment device is adapted and configured to increase an amount of oxygen flowing into the tubing inlet orifice during an increase in altitude and decrease an amount of oxygen flowing into the tubing inlet orifice during a decrease in altitude.

13. A method of using the oxygen delivery system of claim 8, comprising the step of providing the oxygen delivery system of claim 8 and causing the oxygen distribution device to be worn by a person.

14. The method of claim 13, wherein the oxygen distribution device is a mask and the person is a passenger or crew member of an aircraft.

15. The method of claim 13, wherein the person is a patient receiving oxygen therapy.
